(19) United States
(12) Patent Application Publication
(10) Pub. No.: US 2025/0352763 A1
(43) Pub. Date: Nov. 20, 2025

(54) MEDICAL DEVICES FOR DELIVERING FLUID

(71) Applicants: Boston Scientific Scimed, Inc., Maple Grove, MN (US); Boston Scientific Medical Device Limited, Galway (IE)

(72) Inventors: Rajivkumar SINGH, Pune (IN); Sharath Kumar G, Kanakapura (IN); Deepak Kumar SHARMA, Muzaffarnagar (IN); Nachiket GOLE, Pune (IN); Rosangel Aimee RAMOS ESPINOZA, Boston, MA (US); Ryan V. WALES, Shrewsbury, MA (US)

(73) Assignees: Boston Scientific Scimed, Inc., Maple Grove, MN (US); Boston Scientific Medical Device Limited, Galway (IE)

(21) Appl. No.: 19/206,580

(22) Filed: May 13, 2025

Related U.S. Application Data
(60) Provisional application No. 63/647,794, filed on May 15, 2024.

(51) Int. Cl. A61M 25/00

(52) U.S. Cl. CPC A61M 25/0082; A61M 25/0029; A61M 2202/0007; A61M 2202/04; A61M 2205/583; A61M 2205/8287

(57) ABSTRACT

Disclosed systems relate to medical devices for delivering fluid. In an example, a device includes a catheter having a distal end and a gasket movably positioned within a lumen of the catheter. The gasket includes a distal end having a first diameter, a proximal end having a second diameter that is greater than the first diameter, and a body connecting the distal end and the proximal end. The device also includes a nozzle positioned on the distal end of the catheter.

### CROSS-REFERENCE TO RELATED APPLICATIONS

This application claims the benefit of priority of U.S. Provisional Patent Application No. 63/647,794, filed on May 15, 2024, the entirety of which is incorporated herein by reference.

### TECHNICAL FIELD

Various aspects of this disclosure relate generally to devices for delivering treatment fluids (e.g., viscous fluids) via insertion devices (e.g., endoscopes), specifically via devices inserted through working channels of insertion devices.

### BACKGROUND

Endoscopes or other insertion devices have attained great acceptance within the medical community as they provide a means for performing procedures with minimal patient trauma while enabling the physician to view the internal anatomy of the patient. Numerous endoscopes have been developed and categorized according to specific applications, such as cystoscopy, colonoscopy, bronchoscopy, upper GI endoscopy, and others.

An endoscope usually has an elongated tubular shaft, having an imaging device (e.g., a video camera or a fiber optic lens assembly) at its distal end. Various surgical tools and instruments may be inserted through a working channel in the endoscope for performing different surgical procedures. For example, treatment devices and/or fluids may be delivered via the working channel.

Traditional approaches to delivering treatment fluids (e.g., hemostatic gel(s)) via an endoscope often suffer from deficiencies. For example, many of these treatment fluids are highly viscous, which may pose problems in delivery. For instance, given this viscosity, existing delivery devices may require such force that multiple applications or deliveries of treatment fluid (e.g., via a syringe) are often needed.

Furthermore, the treatment fluid may set or cure (e.g., at least partially harden) within the working channel or within a lumen of the treatment device. In some cases, a large amount of time may be required to complete delivery, resulting in delays of other parts of necessary procedures. In other cases, additional treatment fluid may be required to compensate for treatment fluids stuck or otherwise positioned in a lumen of the delivery device. Aspects of this disclosure address these concerns.

### SUMMARY

Aspects of the disclosure relate to, among other things, systems, devices, for devices for delivering fluids to a treatment site. An exemplary delivery device uses a gasket within a catheter to deliver the treatment fluid to a patient.

In some aspects, the techniques described herein relate to a device for delivering a treatment fluid, the device may include a catheter, a gasket, and a nozzle. The catheter may include a distal end. The gasket may be movably positioned within a lumen of the catheter. The gasket may include a distal end having a first diameter, a proximal end having a second diameter that is greater than the first diameter, and a body connecting the distal end and the proximal end. The nozzle may be positioned on the distal end of the catheter.

In some aspects, the gasket may include one or more curved surfaces on the proximal end and/or the distal end.

In some aspects, the device may further include a syringe. The catheter may include a proximal end, and the proximal end of the catheter may be connected to the syringe.

In some aspects, the syringe may include a plunger, which when depressed, may cause a fluid disposed within the syringe to enter the proximal end of the lumen, the fluid to displace the gasket in a distal direction, and the gasket to cause a treatment fluid to be delivered from the nozzle.

In some aspects, the device further may include a tube connected to the syringe, inserted into the proximal end of the lumen, and connected to the gasket, wherein the gasket is a balloon gasket that is inflatable via the tube.

In some aspects, when in an expanded state, the gasket may be generally cylindrical.

In some aspects, the plunger is a first plunger, the syringe may include a second plunger, and distal movement of the second plunger relative to the first plunger is configured to deliver a fluid through the tube to inflate the balloon gasket.

In some aspects, the syringe may include a tip and wherein the tube is movable within the tip.

In some aspects, the catheter may include an inlet positioned in a proximal portion of the lumen.

In some aspects, the syringe may be a first syringe, and the device may include a second syringe coupled to the inlet.

In some aspects, the second syringe may be configured to deliver a fluid into the catheter via the inlet to move the gasket distally.

In some aspects, the techniques described herein relate to a device, the tube may include a tube lumen, the gasket may include a gasket lumen, and the tube lumen may be fluidly connected to the gasket lumen via one or more outlets.

In some aspects, the techniques described herein relate to a device, further including a seal positioned within a portion of the catheter and a tube that may be movably positioned through the seal.

In some aspects, the techniques described herein relate to a device, in which the syringe further may include one or more markings on a body of the syringe that indicate an amount of fluid to dispense to cause the gasket to urge the treatment fluid from the lumen.

In some aspects, the techniques described herein relate to a device, in which the catheter may include an additional lumen and an additional gasket is movably positioned within the additional lumen.

In some aspects, the techniques described herein relate to a device for delivering a treatment fluid via an endoscope, the device may include a catheter including a distal end, a first lumen, and a second lumen. Each lumen may extend along a longitudinal axis of the catheter. The device may also include a first gasket movably positioned within the first lumen. The device may further include a second gasket movably positioned within the second lumen. The device may include a nozzle positioned on the distal end of the catheter.

In some aspects, the techniques described herein relate to a device, further including at least one syringe. The catheter may include a proximal end, and the proximal end of the catheter may be connected to the at least one syringe.

In some aspects, the techniques described herein relate to a device, and the at least one syringe may include a plunger When depressed, the plunger may cause a fluid disposed within the syringe to enter the proximal end of one or more of the first lumen and the second lumen and to displace one or more of the first gasket and the second gasket.

In some aspects, the techniques described herein relate to a device for delivering a treatment fluid. The device may include a catheter having a lumen a distal end; a coil wound radially around or within a wall of a portion of the catheter; and a magnetic gasket movably positioned within the lumen of the catheter. The gasket may include a distal end having a first diameter, a proximal end having a second diameter that is greater than the first diameter, and a body connecting the distal end and the proximal end. When an electric current is applied to the coil, the electric current may generate a magnetic field that causes the magnetic gasket to move within the catheter.

In some aspects, the techniques described herein relate to a device, and when an electric voltage is applied to the coil, an electric current may be caused to flow through the coil, causing a magnetic field to urge the magnet gasket along a longitudinal axis of the lumen.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIGS. 1A-1B depict a delivery device for delivering a treatment fluid, including an enlarged view of a distal portion of the delivery device, according to aspects of this disclosure.

FIG. 2 depicts a perspective view of a gasket of the delivery device of FIGS. 1A-1B, according to aspects of this disclosure.

FIGS. 3A-3C depict views of operation of another exemplary delivery device for delivery of a fluid, according to aspects of this disclosure.

FIGS. 4A-4D depict a delivery device for delivering a treatment fluid, according to aspects of this disclosure.

FIG. 5 depicts a proximal portion of a delivery device for delivering a treatment fluid, according to aspects of this disclosure.

FIG. 6 depicts a delivery device for delivering two treatment fluids, with the delivery device having two gaskets, according to aspects of this disclosure.

FIG. 7 depicts a delivery device for delivering a treatment fluid having a magnetic gasket, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. Proximal and distal directions are labeled with arrows marked “P” and “D”, respectively, throughout various figures. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Aspects of this disclosure relate to devices for delivering fluids via devices, for example, delivered through endoscopes, specifically via working channels (inner channels) of endoscopes. A working channel of an endoscope generally extends from a proximal end (e.g., near the operator of the endoscope) through to a distal end (first placed into a patient's body).

The following non-limiting example is introduced for discussion purposes. A delivery device is used to deliver a treatment fluid to a patient. The delivery device includes a catheter that may be delivered to a treatment site, for example, via a working channel of an endoscope. Additionally, the delivered device includes a movable gasket within the catheter. A treatment fluid is transferred to a distal end of the catheter, for example, distal of the movable gasket. A nozzle or other opening is then positioned on the distal end of the catheter, which is then placed into the proximal end of the working channel of the endoscope and routed through the working channel.

Continuing the example, an operator may then cause pressure to be exerted at the proximal end of the catheter, for example, by attaching a syringe to the proximal end and applying force to a plunger of the syringe. In turn, a fluid (e.g., a liquid or gas) urges the gasket towards the distal end of the catheter, which in turn urges the treatment fluid out of the distal end of the catheter (e.g., through the nozzle) and onto a treatment site within the patient. In one example, the treatment fluid is a hemostatic agent or gel that helps stop or prevent bleeding from a treatment site, such as an ulcer. Similarly, treatment fluid(s) may be applied to areas of the stomach, esophagus, colon, other portions of the gastrointestinal (GI) tract, or other body lumens. One example of a fluid is fibrin.

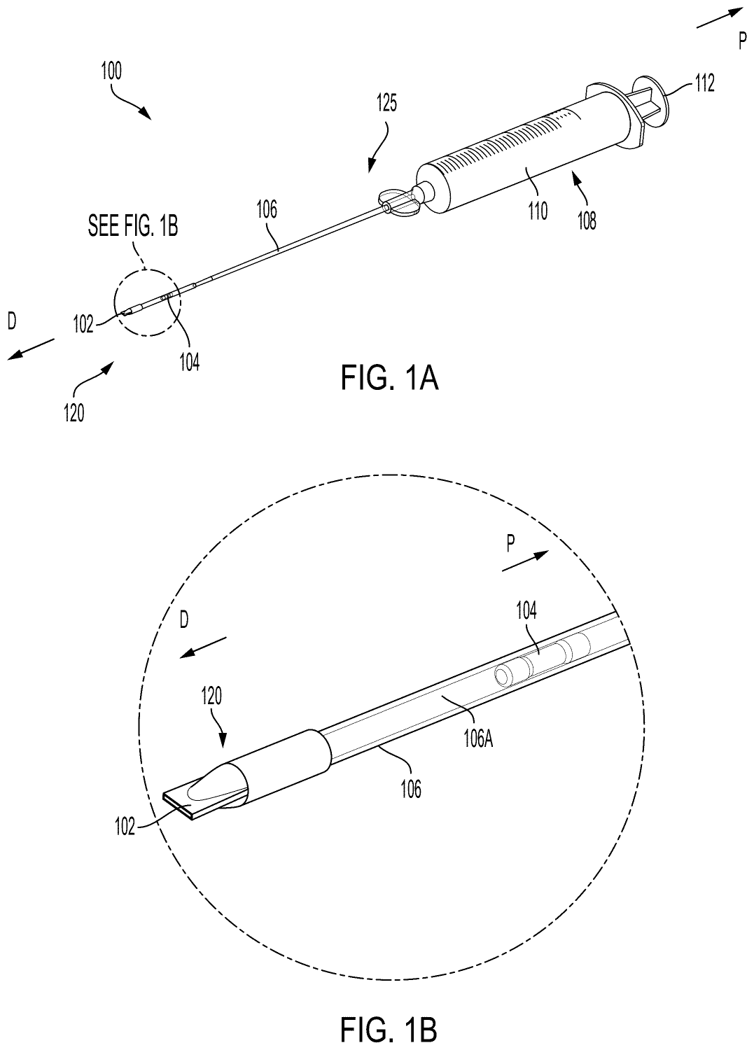

Turning now to the Figures, FIGS. 1A-B depict an exemplary delivery device 100 for delivery of a treatment fluid, for example, through a working channel of an endoscope, according to aspects of this disclosure. FIG. 1A is a first view of a delivery device 100, whereas FIG. 1B includes an enlarged view of a distal portion of delivery device 100.

As explained further herein, the delivery devices disclosed herein, such as delivery device 100, may be used to deliver treatment fluids to a patient via an endoscope or a similar device. A variety of materials may be delivered with delivery device 100. For example, treatment fluids may include any compound or agent used for a diagnosis, a treatment, and/or palliation of a patient's disease or injury. Treatment fluids may include one or more hemostatic material(s) or agent(s). Additionally or alternatively, a treatment fluid may be a first part of a multi-part material (e.g., a two-, three-, or four-part material). For example, once a first treatment fluid is combined with a second treatment fluid, the combined fluid may undergo a reaction (e.g., coagulation or other chemical reaction). In this case, any additional part(s) of the multi-part material may be delivered via a delivery device or another means.

An endoscope may be part of a system. For example, an endoscope system may include an endoscope and various system components such as a controller, a light source, an imaging device, a source of suction and/or irrigation, etc. The endoscope may include an umbilicus that can provide fluid for irrigation from the water/fluid supply and/or suction to a distal tip of a shaft of the endoscope. In some aspects, the light source and/or imaging device of the endoscope may help the user position the distal portion of delivery device 100 relative to the treatment site.

Additionally, in other aspects, the delivery devices disclosed herein may be delivered to a treatment site via a different insertion device, for example, via a cholangioscope, bronchoscope, ureteroscope, duodenoscope, gastroscope, endoscopic ultrasonography (“EUS”) scope, colonoscope, laparoscope, arthroscope, cystoscope, aspiration scope, sheath, or catheter, among other examples. Alternatively, delivery device 100 may be delivered to a treatment site without a use of an insertion device.

As depicted, delivery device 100 includes a nozzle 102, a gasket 104, a tube or a catheter 106, and a control device, for example, a syringe 108. Delivery device 100 may have additional components. Further, some components may be omitted from some aspects.

Catheter 106 is a tube with at least one lumen 106A (FIG. 1B), for example, that extends along a longitudinal axis of device 100. In an example, catheter 106 has an external diameter that compatible with, for example, smaller than, 10.5-12 mm diameter (e.g., the diameter of the working channel) endoscopes. But different diameters are possible. A portion (e.g., a distal portion) of catheter 106 may store one or more treatment fluids to be delivered to a treatment site, but may contain other fluids to aid delivery (e.g., one or more other liquids, gels, air, and/or other gases). Catheter 106 has a distal end 120 and a proximal end 125.

Nozzle 102 is positioned at distal end 120 of catheter 106. As depicted, nozzle 102 is a “duck bill” nozzle. But other types of nozzles or openings may be used, such as flat fan, hollow cone, solid stream, etc. Nozzle 102 may help to allow for the delivery of one or more treatment fluids from catheter 106, for example, when pressure is applied to the one or more treatment fluids, for example, via gasket 104 and/or syringe 108. Further, nozzle 102 may help to prevent treatment fluid(s), other fluids, etc. from the treatment site from flowing proximally into catheter 106. Additionally, in some aspects, nozzle 102 may help to prevent the treatment fluid(s) within catheter 106 from curing, setting, solidifying, or otherwise changing properties before the treatment fluid(s) are delivered to the treatment site.

In some embodiments, nozzle 102, catheter 106, and/or syringe 108 may be attachable or detachable from each other. For example, catheter 106 may be configured to couple/decouple from syringe 108. For example, nozzle 102 may be attachable to, and removable from, catheter 106, for example, to facilitate placement of treatment fluid in the catheter 106. In some cases, nozzle 102 may be affixed, for instance, a friction fit, or snap fit, to catheter 106. Alternatively, nozzle 102 may be a portion of catheter 106, for example, integrally formed with catheter 106. As discussed further herein, delivery device 100 may dispense treatment fluid via nozzle 102. Nozzle 102, gasket 104, catheter 106, and/or syringe 108 may be reusable or single-use.

Syringe 108 may be coupled to proximal end 125 of catheter 106. Syringe 108 may be attachable to, and removable from, catheter 106, for example, via a friction or snap fit. Alternatively, syringe 108 and proximal end 125 of catheter 106 may each have threading, for example, to engage with respective portions to couple syringe 108 (e.g., a distal portion of syringe 108) to proximal end 125 of catheter 106. While a syringe is depicted, other fluid sources may be used. Examples include bulbs, electric pumps, hand pumps, foot pumps or other pedals, tubes, etc.

In some cases, syringe 108 may contain and dispense a fluid, such as a liquid or gas (e.g., air) into catheter 106. Syringe 108 is used to apply a force to urge a fluid contained within syringe 108 and/or a portion of catheter 106. The urged fluid may in turn urge gasket 104 distally, for example, to help urge treatment fluid towards the distal end 120 of the catheter 106 and out of nozzle 102.

For example, in some aspects, syringe 108 includes a plunger 112 and a stationary body 110. Stationary body 110 may be fixedly coupled to proximal end 125 of catheter 106. Stationary body 110 may include one or more markings or other indications and/or may be at least partially transparent.

Distal movement of plunger 112 relative to stationary body 110 may urge one or more fluids (e.g., within stationary body 110 and/or catheter 106) distally into and/or through catheter 106. The markings or indications on stationary body 110 may indicate a volume of fluid and may enable correlation of a position of distal portion of plunger 112 relative to markings/indications to an amount of treatment fluid delivered from nozzle 102. In some cases, a distal portion of the plunger 112 of the syringe 108 may include rubber or other flexible material to help urge the air/fluid distally while also retaining a proximal seal within syringe 108.

When plunger 112 is depressed or urged distally, a fluid (distinct from the treatment fluid) is pushed through stationary body 110 into catheter 106. The fluid in turn urges gasket 104 distally within lumen 106A of catheter 106. In turn, the gasket 104 urges the treatment fluid to transition down the catheter 106 from the proximal end 125 to the distal end 120 and ultimately to be delivered via nozzle 102.

Movement of the plunger 112 relative to stationary body 110 may urge the fluid to help control of a position of gasket 104 within the catheter 106. For example, plunger 112 may be moved proximally to pull or retract the treatment fluid proximally within the lumen 106A, for example to help prevent inadvertent delivery of treatment fluid. Alternatively, plunger 112 may be moved distally to urge gasket 104 distally within the lumen 106A, and correspondingly urge the treatment fluid distally within the lumen 106A. Additionally, movement of plunger 112 may help control the delivery of the treatment fluid due to the frictional engagement of gasket 104 with the inner surface of the lumen 106A, frictional resistance of nozzle 102, a viscosity of the treatment fluid, etc.

Nozzle 102, gasket 104, catheter 106, and/or syringe 108 may each be manufactured with one or more biocompatible materials. Catheter 106 may be formed of a flexible material such as a flexible plastic or PVC, such that catheter 106 may bend through the working channel of the endoscope as the endoscope traverses a body lumen (e.g., the GI tract) as necessary. Syringe 108 may be formed of a harder plastic.

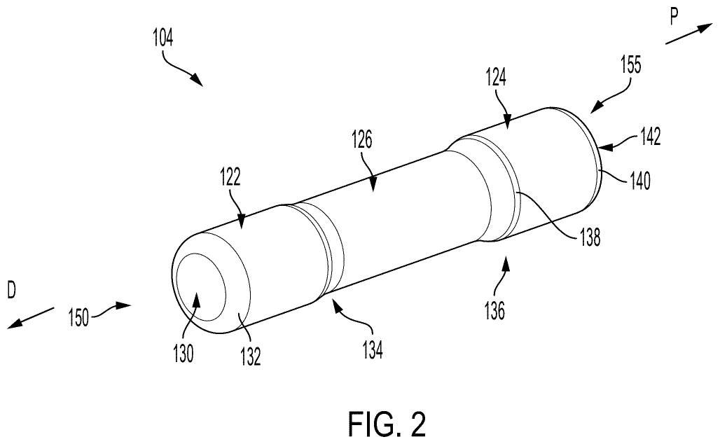

Gasket 104 may be inserted or removed from catheter 106. For example, gasket 104 may be initially positioned at a middle or intermediate position within catheter 106 and may be free to move along the longitudinal axis of catheter 106. There may be some friction between an inner surface of lumen 106A within catheter 106 and gasket 104. In these aspects, gasket 104 is not free floating within lumen 106A of catheter 106. Gasket 104 may be inserted to or removed from catheter 106 as necessary. Gasket 104 serves to aid in the delivery of the treatment fluid to a patient. Gasket 104 is shown in more detail in FIG. 2.

FIG. 2 depicts a perspective view of gasket 104 of delivery device 100. As discussed, gasket 104 may be used to urge treatment fluid through lumen 106A of catheter 106. Gasket 104 has multiple contact surfaces, for example, that contact the inner surface(s) of lumen 106A of catheter 106 as gasket 104 moves within lumen 106A. As depicted, gasket 104 includes a proximal or front contact surface 122, a distal or rear contact surface 124, and a body 126.

Gasket 104 includes a distal end 150 and a proximal end 155. Front contact surface 122 and rear contact surface 124 each may contact with an inner surface of the lumen 106A of catheter 106. Body 126 connects front contact surface 122 and rear contact surface 124.

Gasket 104 includes various other surfaces. For example, gasket 104 includes a front flat surface 130 positioned on distal end 150, a front distal curved surface 132, and a front proximal curved surface 134, each of which is positioned distally relative to body 126. For example, front distal curved surface 132 and front proximal curved surface 134 may be positioned on opposing ends of front contact surface 122. Gasket 104 further includes a rear distal curved surface 136, a rear proximal curved surface 138, rear proximal curved surface 140, and rear flat surface 142. Front proximal curved surface 134 and rear distal curved surface 136 may each be formed of two pieces or two portions with different curvatures, as depicted. In some cases, while various separate surfaces are described, for example, made in two or more separate pieces and assembled, two or more of these surfaces may be formed by a single piece of material.

As explained below, distal end 150 is typically oriented towards the distal end 120 of catheter 106 and that proximal end 125 is typically oriented towards the proximal end 155 of the catheter 106. Rear contact surface 124 has a diameter greater than a diameter of front contact surface 122. In some cases, the diameters of the components of gasket 104 may decrease between front and rear. For example, one or more portions of body 126 may have a diameter that is less than the diameter of both front contact surface 122 and rear contact surface 124. Body 126 may help to maintain longitudinal orientation of gasket 104, for example, as gasket 104 moves longitudinally within catheter 106, especially if catheter 106 is bent and/or curved, for example, through a tortuous portion of body lumen.

The size and shape of gasket 104 may help to maintain an alignment of gasket 104 within catheter 106, for example, given a curvature of catheter 106 within the working channel of the endoscope. In some cases, some treatment fluid may flow around one or more portions of gasket 104, e.g., from the distal end in the proximal direction. Nevertheless, the size and/or shape of gasket 104 may help to minimize an amount of the treatment fluid that may flow around one or more portions of gasket 104. Additionally, some treatment fluid being positioned between gasket 104 and the inner surface of lumen 106A of catheter 106 may help to lower friction between gasket 104 and catheter 106. Alternatively or additionally, one or more coatings or layers (e.g., one or more hydrophilic coatings or layers, for example, Polytetrafluoroethylene (PTFE)) may be positioned around one or more exterior portions of gasket 104. The one or more coatings or layers may help gasket 104 move (e.g., slide) within lumen 106A of catheter 106.

In some cases, a surface area of rear contact surface 124 may be greater than a surface area of front contact surface 122, for example, because rear contact surface 124 may have a diameter greater than the diameter of front contact surface 122. As depicted, front contact surface 122 and rear contact surface 124 may each have flat or approximately flat surfaces that are parallel to a longitudinal axis of the gasket. In some cases, however, a surface of front contact surface 122 may be at least partially conical, for example, with a proximal portion of front contact surface 122 being larger (e.g., in diameter) than a distal portion of front contact surface 122. The at least partially conical shape may help reduce the force (e.g., distal force on gasket 104) required to move gasket 104 distally within catheter 106 and apply the treatment fluid. Similarly, although not shown, rear contact surface 124 may include an at least partially conical shape as well.

As discussed further herein, the treatment fluid may interact with one or more front-facing surfaces of gasket 104, such as front flat surface 130, front curved surface 132, front contact surface 122, etc. Similarly, air or other (non-treatment) fluid may interact with one or more rear facing surfaces, such as proximal rear distal curved surface 138, rear proximal curved surface 140, rear contact surface 124, and rear flat surface 142.

The varying diameters of gasket 104 and the curved surfaces helps reduce or eliminate friction points as treatment fluid is pushed through catheter 106. In some cases, some treatment fluid may flow around the front contact surface 122, given the smaller diameter. This may help to help ensure the proper alignment of gasket 104, for example, if catheter 106 is curved or bent due to device 100 being positioned within an endoscope, which may itself be bent in places.

In operation, front contact surface 122 is positioned facing distal end 120 of catheter 106, and rear contact surface 124 is positioned facing towards proximal end 125 of catheter 106. In an example use case, gasket 104 may be positioned within catheter 106, with front contact surface 122 oriented towards distal end 120 of catheter 106.

Next, the treatment fluid to be delivered to the patient is placed in the catheter 106, specifically, via distal end 120. As treatment fluid enters catheter 106, the treatment fluid urges gasket 104 proximally toward proximal end 125 of the catheter 106. When the treatment fluid is completely delivered to catheter 106, nozzle 102 is placed on distal end 120, which helps to form a partial seal. At this point, air and/or another fluid may be in catheter 106, proximal to gasket 104.

Next, a proximal portion of catheter 106 is connected to syringe 108.

Syringe 108 may be contain air, water, or another fluid or gas. The catheter assembly, which includes catheter 106 and nozzle 102 (and gasket 104 within catheter 106) is placed within a working channel of an endoscope, with distal end 120 being delivered through the working channel first. Syringe 108 is attached to the proximal end 125 of catheter 106, and syringe 108 remains proximal of the endoscope such that the user may manipulate plunger 112 relative to stationary body 110. A volume of catheter 106 may be less than a volume of syringe 108. Alternatively, the volume of catheter 106 may be greater than the volume of syringe 108, for example, if a volume of air or gas is positioned within catheter 106 proximal of gasket 104.

Next, to deliver the treatment fluid to a patient, a force is applied to plunger 112 of syringe 108. The force pushes the air or additional fluid towards gasket 104, which in turn pushes the treatment fluid towards distal end 120 and distally from nozzle 102, delivering the treatment fluid to the patient. In some cases, the treatment fluid may be cured via a presence of light, such as ultraviolet light applied near the distal end of the endoscope. The ultraviolet light may be emitted from one or more illumination or light sources at the distal end of the endoscope (not shown). Further, in an aspect, a liquid substance, such a lubricant, may be positioned on an inside (e.g., within lumen 106A) of catheter 106 before use. The substance may help gasket 104 and/or the fluid(s) (e.g., the treatment fluid(s) and/or the fluid(s) delivered to catheter 106 via syringe 108) move longitudinally within catheter 106.

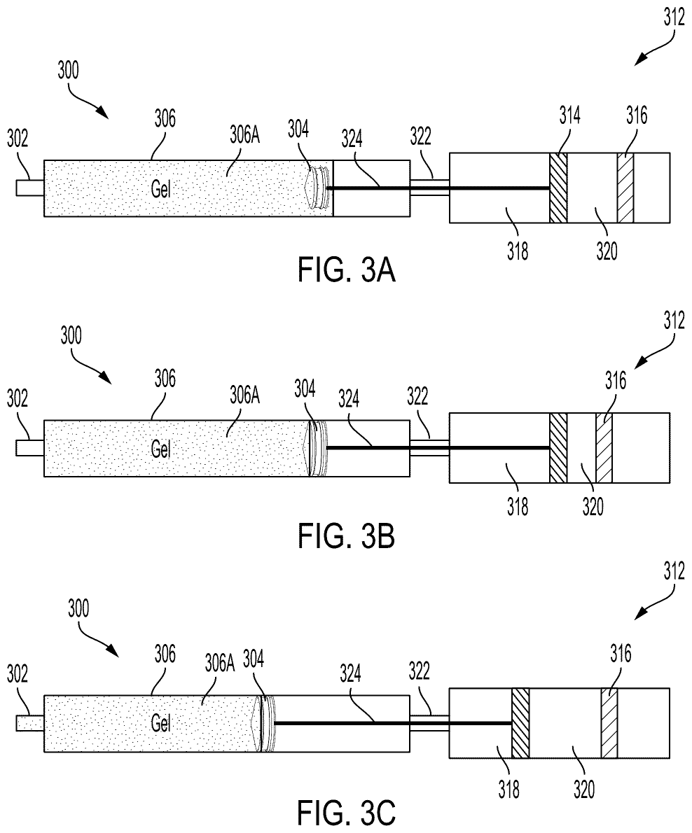

In some aspects, gasket 104 may be an expandable gasket or a balloon gasket. In these aspects, the gasket is inflated as part of a delivery process, as explained further in FIGS. 3A-C.

FIGS. 3A-3C depict views of operation of another exemplary delivery device for delivery of a fluid (e.g., a treatment fluid), according to aspects of this disclosure. FIGS. 3A-3C depict a delivery device 300 and a handle portion 312. Handle portion 312 may be connected to delivery device 300 via a tube 324.

Delivery device 300 includes a nozzle 302, a catheter 306, and an expandable or balloon gasket 304. Examples of nozzle 302 include nozzle 102 discussed above. Catheter 306 is a tube with at least one lumen 306A, for example, that extends partially along a longitudinal axis of catheter 306. Balloon gasket 304 is inflatable or expandable, for example, when fluid (e.g., liquid or air) is urged into balloon gasket 304. When expanded, balloon gasket 304 is generally cylindrical in shape. In some cases, balloon gasket 304 may also be deflated, for instance, when air the fluid is removed from balloon gasket 304.

Handle portion 312 includes at least two actuators or plungers, for example, a distal plunger 314 and a proximal plunger 316. Distal plunger 314 and proximal plunger 316 together with handle portion 312 form at least two cavities, for example, a first cavity 318 and a second cavity 320, within handle portion 312. Movement of distal plunger 314 controls the movement of a first fluid, and movement of proximal plunger 316 controls the movement of a second fluid. Both distal plunger 314 and proximal plunger 316 are moveable within handle portion 312, but also each help to form a seal between a radial exterior of the respective plunger and an inner diameter of the handle portion 312.

First cavity 318 (e.g., controlled by distal plunger 314) is fluidly connected to balloon gasket 304 via one or more tubes (channels) 324, for example, extending through a portion of second plunger 316. A distal movement of distal plunger 314 may urge air or fluid distally, through the one or more tubes 324, to balloon gasket 304. The air/fluid may inflate, expand, or otherwise enlarge balloon gasket, for example, between a first configuration shown in FIG. 3A and a second configuration shown in FIG. 3B. As depicted, tube 324 is positioned within a tip 322 of plunger. But in some cases, tip 322 may not be present, and tube 324 connect directly to handle portion 312.

Proximal plunger 316 may be fixedly coupled to balloon gasket 304 via the one or more tubes 324. In these aspects, distal movement of proximal plunger 316 may also urge balloon gasket 304 distally.

Alternatively or additionally, a second air or fluid may be in second cavity 320, e.g., distal of second plunger. Distal movement of proximal plunger 316 may urge the second air or fluid distally, which in turn urges the balloon gasket 304 distally, as discussed above with respect to FIGS. 1 and 2.

In the example depicted, FIG. 3A depicts an initial position of balloon gasket 304. Tube 324 is connected from distal plunger 314 to balloon gasket 304. As depicted in FIG. 3B, proximal plunger 316 is moved distally, which helps to inflate gasket 304, for example, with air, thereby expanding gasket 304. This results in a tighter seal between the balloon gasket 304 and the inner surface of the lumen within catheter 306. Continuing the example, as depicted in FIG. 3C, distal plunger 314 is moved distally, which urges expanded balloon gasket 304 distally, which urges the treatment fluid distally within catheter 306 and out of nozzle 302 to the treatment site.

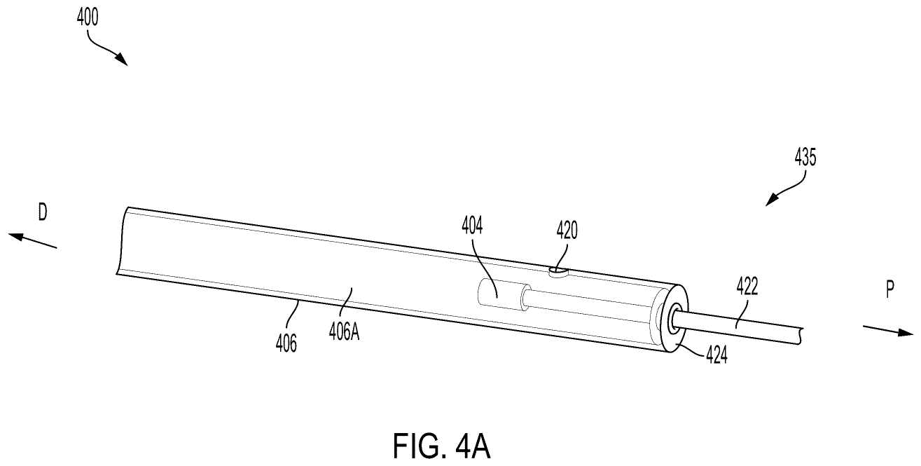

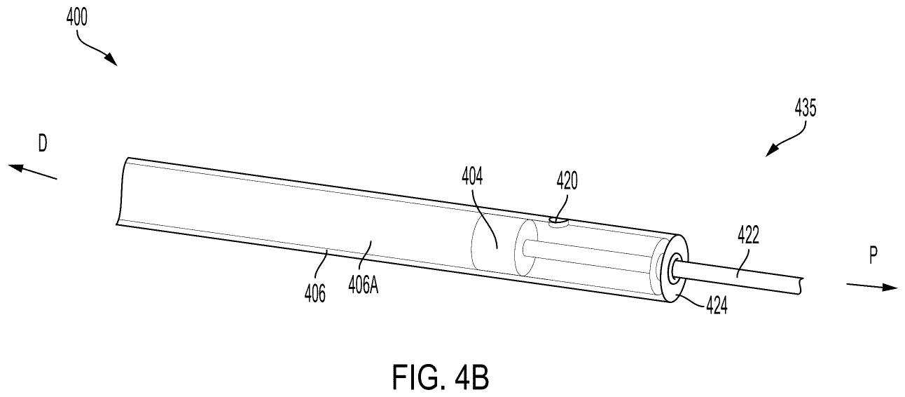

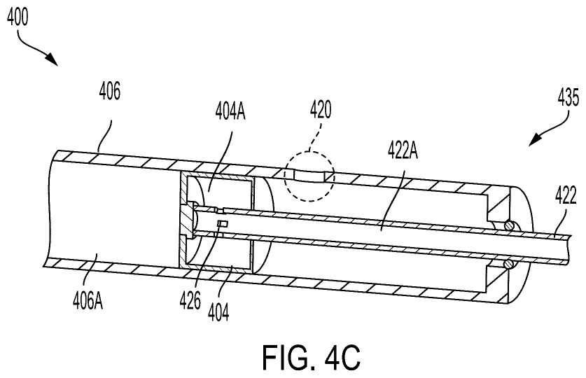

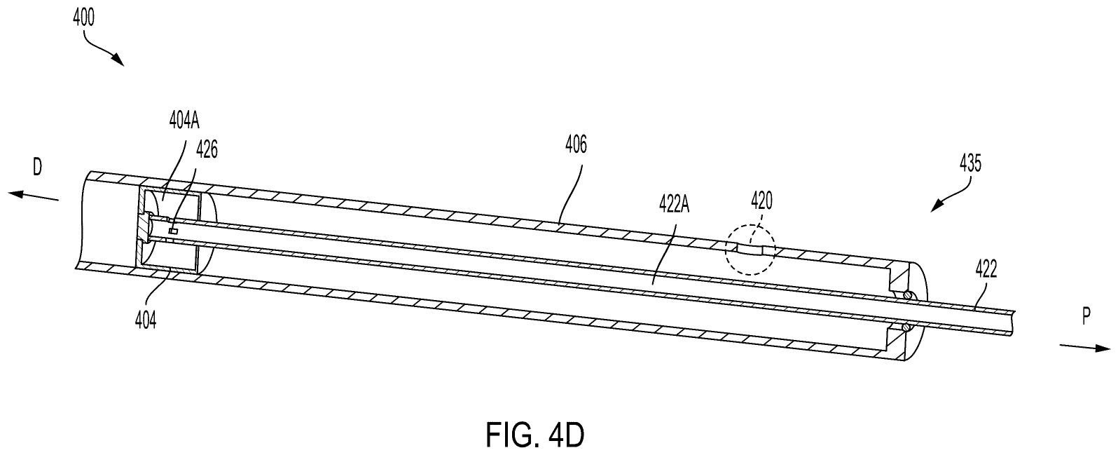

FIGS. 4A-4D depict portions of a delivery device 400 for delivering a treatment fluid, according to aspects of this disclosure. Delivery device 400 includes a catheter 406, an expandable or balloon gasket 404, a tube 422, an inlet 420, and a seal or gasket 424 (“gasket seal 424”). Delivery device 400 may have other components. Further, some components may be omitted from some aspects.

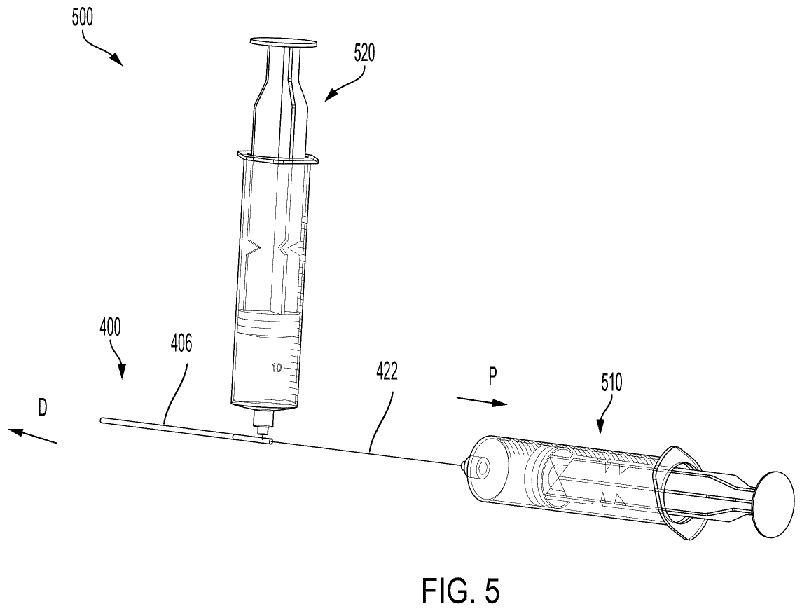

Catheter 406 is a tube with at least one lumen 406A, for example, that extends along a longitudinal axis of device 400. Catheter 406 has a distal end (not depicted) and a proximal end 435. Although not shown, a nozzle, for example, nozzle 102, may be positioned at the distal end. One or more syringes (FIG. 5) may be coupled to proximal end 435. Tube 422 is connected on one end to balloon gasket 404 and on another end to a handle portion, or proximal control device, e.g., including a syringe. A portion of tube 422 is movably positioned within lumen 406A of catheter 406, for example, extending through gasket seal 424.

Urging air through tube 422 may cause balloon gasket 404 to expand. Gasket seal 424, positioned at proximal end 435 of catheter 406, helps to seal proximal end 435 of catheter 406 to prevent any leakage (e.g., of air, liquid, or other fluid). In an aspect, tube 422 is slideable (e.g., longitudinally movable) through gasket seal 424, while maintaining a seal around tube 422. Delivery device 400 may be used similarly as delivery device 100, discussed above.

FIG. 4A depicts a first configuration of delivery device 400. As depicted in FIG. 4A, balloon gasket 404 is not inflated. Fluid flow through tube 422 may help to expand balloon gasket 404. For example, as shown in FIGS. 4C and 4D, tube 422 may include a tube 422 that is fluidly connected to a gasket lumen 404A via one or more outlets 426.

FIG. 4B depicts a second configuration of delivery device 400. As depicted in FIG. 4B, balloon gasket 404 is inflated. Once the balloon gasket 404 is expanded, one or more fluids (e.g., treatment agent(s) can be positioned within lumen 406A, for example, via the distal end of catheter 406, as discussed above with respect to FIG. 2. Alternatively, one or more fluids may be positioned within lumen 406A before inflation of balloon gasket 404. In any of these aspects, catheter 406 includes inlet 420, for example, in an outer wall of catheter 406 at a position proximal of gasket 404 when gasket 404 is inflated or otherwise expanded. In these aspects, gasket 404 may divide lumen 406A of catheter 406 into a proximal portion (e.g., proximal of gasket 404) and a distal portion (e.g., distal of gasket 404). Gasket 404 may help to at least partially the proximal portion of lumen 406A from the distal portion of lumen 406A.

FIG. 4C depicts a first longitudinal cross-section of delivery device 400. As depicted in FIG. 4C, balloon gasket 404 is distal of Inlet 420. In some aspects, one or more fluids may be introduced the proximal portion of lumen 406A via inlet 420, which may help to urge gasket 404 distally to deliver one or more treatment agents positioned in the distal portion of lumen 406A. Alternatively or additionally, in some aspects, gasket 404 may be longitudinally movable based on longitudinal movement of tube 422. For example, a user may urge tube 422 distally to urge gasket 404 distally in order to deliver one or more treatment agents positioned in the distal portion of lumen 406A. In some aspects, inlet 420 may help to allow air into the proximal portion of lumen 406A of catheter 406, which may help to reduce a pressure required to urge the treatment fluid out of the distal end of catheter 406. More specifically, because the gasket seal 424 seals the proximal side of catheter 406, the fluid flow through inlet 420 may help to urge gasket 404 distally.

FIG. 4D depicts a second longitudinal cross-section of delivery device 400. Relative to FIG. 4C, gasket 404 is extended further to the distal end of catheter 406. The seal in proximal end 435 of catheter 406 is maintained by gasket seal 424. Additionally, the movement of gasket 404 may help to deliver one or more treatment fluids from lumen 406A of catheter 406.

FIG. 5 depicts a proximal portion of a delivery system 500 for delivering a treatment fluid, according to aspects of this disclosure. FIG. 5 depicts delivery device 400, a first syringe 510, and a second syringe 520. As discussed above, delivery device 400 includes catheter 406 and tube 422. First syringe 510 may be coupled to tube 422. In this aspect, first syringe 510 may be used to deliver one or more fluids to help expand gasket 404 (FIGS. 4A-4D). Second syringe 520 may be coupled to catheter 406, for example, via inlet 420. Second syringe 520 may be used to deliver one or more fluids to help push or urge gasket 404 (FIGS. 4A-4D) distally to help deliver one or more treatment fluids.

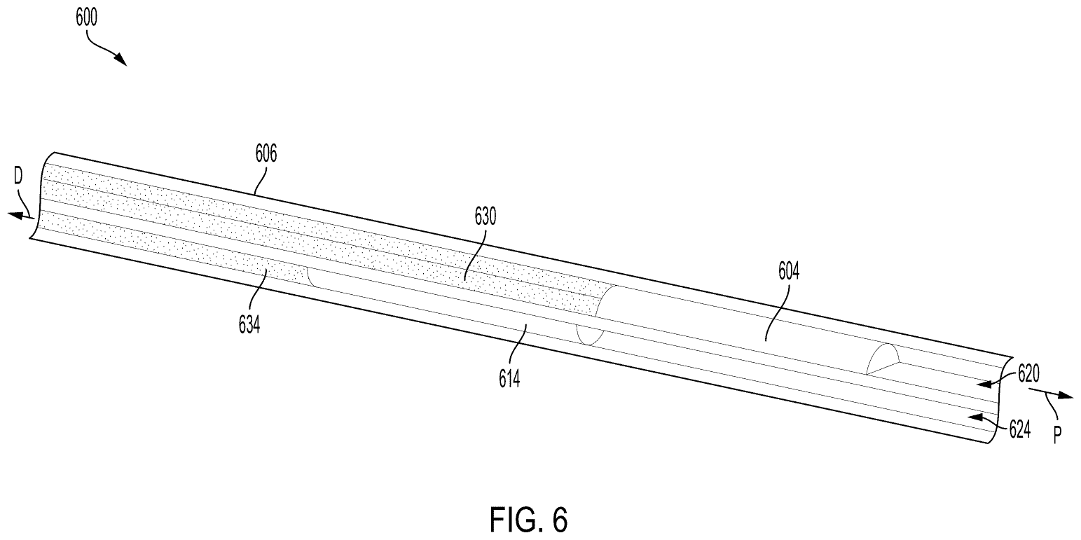

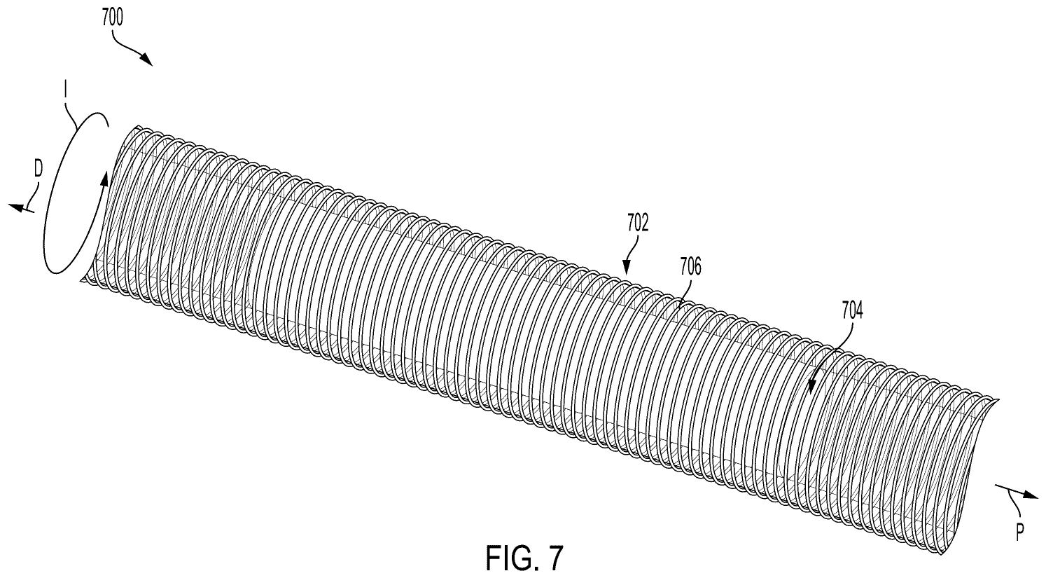

FIG. 6 depicts a delivery device for delivering two treatment fluids, with the delivery device having two gaskets. FIG. 6 depicts a delivery device 600 with a catheter 606 having a first lumen 620, a second lumen 624, a first gasket 604, and a second gasket 614.

Relative to delivery devices 100 and 300, delivery device 600 may receive and deliver two agents, e.g., first agent 630 and second agent 634, simultaneously or in series. As depicted, gasket 604 is positioned within first lumen 620, and gasket 614 is positioned within second lumen 624. While two lumen are depicted, additional chambers are possible.

Movement of first gasket 604 may help to deliver agent 630, and movement of second gasket 614 may help to deliver agent 634. Techniques as discussed with respect to delivery device 100 may be used. For example, first agent 630 may be provided into a distal end of lumen 620, and second agent 634 in a distal end of lumen 624. A nozzle may be placed on the distal end of delivery device 600. Then, a fluid may be urged into a proximal end of lumen 620, causing gasket 604 to urge agent 630 to exit catheter 606. At the same time or at a different time, a fluid may be urged into a proximal end of lumen 624, and then the second gasket 614 may urge second agent 634 to exit catheter 606. Although not shown, in some aspects, a distal portion of catheter 606 may include a mixing chamber, for example, such that first agent 630 and second agent 634 may mix in the mixing chamber before being delivered out of catheter 606 or otherwise applied to the treatment site. The dual gasket configuration may help to facilitate a curing gel to cure when the two agents 630 and 634 mix or otherwise make contact at a treatment site. Additionally, because the two agents 630 and 634 do not mix within a proximal portion of catheter 606, there may be a reduced likelihood of the two agents 630 and 634 forming one or more clogs within catheter 606.

In another configuration, agents 630 and 634 may be delivered with a single gasket. For example, agents 630 and 634 may arranged longitudinally or serially within a single lumen of catheter 606. In some aspects, one or more fluids (e.g., air, saline, or other fluid) may be positioned (e.g., trapped) between agents 630 and 634. For instance, in this configuration, distal movement of the gasket may first cause first agent 630 to be delivered. Further distal movement of the gasket may deliver the separation fluid, and then additional distal movement of the gasket may cause second agent 634 to be delivered.

FIG. 7 depicts a delivery device for delivering a treatment fluid having a magnetic gasket, according to aspects of this disclosure. FIG. 7 depicts a portion of a delivery device 700, which includes a coil 702, a magnetic gasket 704, and a catheter 706.

Coil 702 is formed of an electrically conducting material and is wrapped around or positioned with a wall of at least a portion of catheter 706. By applying an electric voltage to coil 702, an electric current may be caused to flow through coil 702 in a first direction I. The electric current in turn creates a magnetic field towards a distal end of delivery device 700. The magnetic field interacts with magnetic gasket 704, which causes the magnetic gasket 704 to move towards the distal end of delivery device 700.

Additionally or alternatively, an electric current through coil 702 in a second direction opposite to the first direction may create a magnetic field toward a proximal end of delivery device 700, which may help to urge magnetic gasket 704 proximally. Coil 702 may be contained within an electrically insulated material within catheter 706, which may help to prevent electricity from traveling outside coil 702.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A device for delivering a treatment fluid, the device comprising:
a catheter having a distal end;
a gasket movably positioned within a lumen of the catheter, wherein the gasket comprises:
a distal end having a first diameter,
a proximal end having a second diameter that is greater than the first diameter, and
a body connecting the distal end and the proximal end; and
a nozzle positioned on the distal end of the catheter.

2. The device of claim 1, wherein the gasket comprises one or more curved surfaces on the proximal end and/or the distal end.

3. The device of claim 1, further comprising a syringe, wherein the catheter comprises a proximal end, and wherein the proximal end of the catheter is connected to the syringe.

4. The device of claim 3, wherein the syringe comprises a plunger, wherein when depressed, the plunger causes a fluid disposed within the syringe to enter the proximal end of the lumen, the fluid to displace the gasket in a distal direction, and the gasket to cause a treatment fluid to be delivered from the nozzle.

5. The device of claim 4, further comprising a tube connected to the syringe, inserted into the proximal end of the lumen, and connected to the gasket, wherein the gasket is a balloon gasket that is inflatable via the tube.

6. The device of claim 5, wherein, in an expanded state, the gasket is generally cylindrical.

7. The device of claim 6, wherein the plunger is a first plunger, wherein the syringe includes a second plunger, and wherein distal movement of the second plunger relative to the first plunger is configured to deliver a fluid through the tube to inflate the balloon gasket.

8. The device of claim 7, wherein the syringe comprises a tip and wherein the tube is movable within the tip.

9. The device of claim 6, wherein the catheter comprises an inlet positioned in a proximal portion of the lumen.

10. The device of claim 9, wherein the syringe is a first syringe, and wherein the device includes a second syringe coupled to the inlet.

11. The device of claim 10, wherein the second syringe is configured to deliver a fluid into the catheter via the inlet to move the gasket distally.

12. The device of claim 11, wherein the tube includes a tube lumen, wherein the gasket includes a gasket lumen, and wherein the tube lumen is fluidly connected to the gasket lumen via one or more outlets.

13. The device of claim 12, further comprising a seal positioned within a portion of the catheter and a tube that is movably positioned through the seal.

14. The device of claim 6, wherein the syringe further comprises one or more markings on a body of the syringe that indicate an amount of fluid to dispense to cause the gasket to urge the treatment fluid from the lumen.

15. The device of claim 1, wherein the catheter includes an additional lumen, and wherein an additional gasket is movably positioned within the additional lumen.

16. A device for delivering a treatment fluid via an endoscope, the device comprising:
a catheter comprising a distal end, a first lumen, and a second lumen, each lumen extending along a longitudinal axis of the catheter;
a first gasket movably positioned within the first lumen;
a second gasket movably positioned within the second lumen; and
a nozzle positioned on the distal end of the catheter.

17. The device of claim 16, further comprising at least one syringe, wherein the catheter comprises a proximal end, and wherein the proximal end of the catheter is connected to the at least one syringe.

18. The device of claim 17, wherein the at least one syringe comprises a plunger, wherein when depressed, the plunger causes a fluid disposed within the syringe to (i) enter the proximal end of one or more of the first lumen and the second lumen and to (ii) displace one or more of the first gasket and the second gasket.

19. A device for delivering a treatment fluid, the device comprising:
a catheter having a lumen a distal end;
a coil wound radially around or within a wall of a portion of the catheter;
a magnetic gasket movably positioned within the lumen of the catheter, wherein the gasket comprises:
a distal end having a first diameter,
a proximal end having a second diameter that is greater than the first diameter, and
a body connecting the distal end and the proximal end, wherein when an electric current is applied to the coil, the electric current generates a magnetic field that causes the magnetic gasket to move within the catheter.

20. The device of claim 19, wherein when an electric voltage is applied to the coil, an electric current is caused to flow through the coil, causing a magnetic field to urge the magnet gasket along a longitudinal axis of the lumen.
